(19) United States
(12) Patent Application Publication
(10) Pub. No.: US 2023/0129312 A1
(43) Pub. Date: Apr. 27, 2023

(54) COMBINED METHOD FOR ESTIMATING INJECTIVITY LOSS IN CARBONATE RESERVOIRS

(71) Applicant: Petróleo Brasileiro S.A. - Petrobras, Rio de Janeiro (BR)

(72) Inventors: Rafaella Magliano Balbi De Faria, Rio de Janeiro (BR); Igor Domingos Rodrigues, Rio de Janeiro (BR); Aline Machado De Azevedo Novaes, Rio de Janeiro (BR)

(21) Appl. No.: 17/970,868

(22) Filed: Oct. 21, 2022

(30) Foreign Application Priority Data
Oct. 21, 2021 (BR) 10 2021 021114 8

(51) Int. Cl. G06F 30/28; G01N 33/24

(52) U.S. Cl. CPC G06F 30/28; G01N 33/24; G06F 2113/08

(57) ABSTRACT

The present invention addresses to a combined experimental and simulation method for estimating the loss of injectivity in carbonate reservoirs subjected to water injection considering both the effects of reactivity and the presence of suspended solids and/or oil, aiming at greater efficiency of water injection. By means of the present invention, it is possible to understand the combined results of rock-fluid interaction and presence of solids in injection fluids and to determine parameters for injectivity loss models, enabling a more realistic prediction for scenarios of carbonate reservoirs.

### FIELD OF THE INVENTION

The present invention addresses to a combined experimental and simulation method for estimating the loss of injectivity of wells, with application in carbonate reservoirs subjected to water injection, considering reactive effects and the presence of suspended solids and/or oil, aiming at a greater efficiency of water injection.

### DESCRIPTION OF THE STATE OF THE ART

Due to the generation of solids in the injection system and, more recently, the possibility of bypassing the Sulfate Removal Units (SRU), to increase injection efficiency, and the reinjection of produced water, due to changes in legislation, it is necessary to carry out studies to predict the loss of injectivity over time. With this prediction, it is possible to estimate, for example, the frequency of operations to restore injectivity and minimize the loss of production caused by the reduction in injection flow rates.

The prediction of loss of injectivity is currently performed from the modeling proposed by PERKINS, T. K. & GONZALEZ, J. A. (1985) “The effect of thermoelastic stresses on injection well fracturing”, Society of Petroleum Engineers Journal, USA, SPE11332. This model considers a pressure increase due to damage formation and presents a parameter, determined experimentally, related to the quality of the injected water and the reservoir rock. This parameter is obtained from flow tests in porous media, carried out in the laboratory or on the platform, or from the adjustment of the history of the injectivity index of a well. The experimental method does not include the use of reactive fluids.

In recent years, tests were carried out with carbonate plugs, in the laboratory and on a platform, following the methodology of flow in porous media validated for sandstone samples. As a reactive fluid was not used, the results were very pessimistic and not consistent with those observed in the field.

In this way, for carbonate reservoirs, it is necessary to consider the interaction between injected fluids and the reservoir. The injection of unbalanced fluids and the presence of CO₂ lead to carbonate dissolution reactions according to the equations below.

CaCO₃+H⁺⇔Ca⁺²+HCO₃⁻; (1)

2CaCO₃+Mg⁺²⇔Ca⁺²+CaMg(CO₃)₂ (2)

The reactive flow tests present an established methodology and their results point to a dissolution of the rock with an increase in permeability and formation of preferential paths.

The reactivity of the system is due to the injection of unbalanced water and the presence of aqueous CO₂ under high pressure conditions. In the reactive flow test methodology, the fluid injection is performed using a bottle with piston displacement. In this methodology, it is not possible to add suspended solids to the fluid that will be injected for two main reasons: (1) there is no agitation in the bottles and this would cause the solids to settle to the bottom, changing the suspended solids content (SSC) along the test; (2) the presence of solids would damage the bottle's functioning, such as metal wear and the piston getting stuck.

In carbonate reservoirs, chemical reactions are expected between the rock and the injected fluids. Parameters such as temperature, pressure, fluid composition, mineralogical composition and amount of CO₂ affect not only the intensity of these reactions but also determine which interactions will be the majority.

These reactions can cause changes in the permoporous properties of the reservoir and in the composition of the produced water, for example. The dissolution of carbonates, such as calcite and dolomite, is expected mainly near the injection well. In this region, part of the CO₂ present in the oil and/or injected passes into the injection water, making it more reactive. This interaction results in an increase in porosity and permeability of the rock, consequently increasing the injectivity index of the well.

Corrosion problems, the possibility of a bypass of the SRU and the need for re-injection of produced water increase the SSC in the injection water. Poorer water quality may cause pores to plug, resulting in greater loss of injectivity in the wells.

In this way, the need arose to develop a methodology that contemplated the reactive flow and the presence of solids in the injected fluid.

Document CN109838220A discloses an evaluation method for a re-injection index of produced water in oil fields, which comprehensively analyzes the quality of produced water and its degree of damage to the permeability and water absorption performance of the reservoir target. It further mentions that the method obtains information on water quality, such as: oil concentration, suspended solids concentration, average diameter of suspended solid particles, concentration of sulfate-reducing bacteria, concentration of saprophytic bacteria and concentration of iron bacteria.

Document BR112013006608A2 discloses an improved production monitoring system to provide advanced control of complex oil and/or gas production systems, comprising a variety of producer and injector wells that operate together in a porous and heterogeneous environment, having the advantage of analyzing the signals from production and injection units in a variety of temporal processes, with different durations, and offers a valuable notion in the operation of production and injection units and, with this, allows production units to and injection are better controlled.

The experimental and simulation methodologies to study these described phenomena (dissolution reactions due to rock-fluid interaction and loss of injectivity caused by solids present in water) exist separately. However, for the scenario of carbonate reservoirs, there is a need of considering the two phenomena together to know which will be predominant one in a given situation, since the dissolution positively to the injectivity of the well and the increase of solids negatively contributes to the same.

In this way, in order to solve such problems, the present invention was developed, by means of an experimental method for reactive fluid injection tests contemplating the presence of solids and a method for simulating the prediction of the loss of injectivity of wells in carbonate reservoir considering both effects: reactivity and presence of solids. By means of this method, it is possible to understand combined results of rock-fluid interaction and presence of solids in injection fluids and to determine parameters for injection loss models, allowing a more realistic prediction for scenarios of carbonate reservoirs.

The present invention has the technical advantages of experimental parameter determination and simulation to estimate the behavior of injectivity in carbonate reservoirs considering reactive effects and the presence of suspended solids. From an economic point of view, it is possible to predict the loss of injectivity of the wells and, with that, more efficiently schedule the intervention to remove damage or even the need of building new wells, avoiding the reduction of injection and, consequently, the reduction of the production from the fields.

### BRIEF DESCRIPTION OF THE INVENTION

The present invention addresses to a combined experimental and simulation method that takes into account the two phenomena together, namely, dissolution reactions due to rock-fluid interaction and loss of injectivity caused by solids present in water, since the dissolution positively contributes and the increase of solids negatively contributes to the injectivity of the well.

The present invention applies to carbonate reservoirs subjected to injection of water with contents of suspended solids and/or oil.

### BRIEF DESCRIPTION OF DRAWINGS

The present invention will be described in more detail below, with reference to the attached figures which, in a schematic way and not limiting the inventive scope, represent examples of its embodiment. In the drawings, there are:

FIG. 1 illustrating a flow system used in reactive tests with the presence of solids according to the present invention;

FIG. 2 illustrating a graph with data of (Q/P)/(Q₀/P₀) over time for all tests performed of reactive flow+SSC;

FIG. 3 illustrating an adjustment graph carried out with the Perkins and Gonzalez model for the test data TF02-2019. The adjusted parameter value was pL=4.25;

FIG. 4 illustrating an adjustment graph carried out with the Perkins and Gonzalez model for the test data TF05-2019, with pL=4.25 and without change in the area open to the flow;

FIG. 5 illustrating an adjustment graph carried out with the Perkins and Gonzalez model for the test data TF05-2019, with pL=4.25 and with a 20% increase in the area open to the flow;

FIG. 6 illustrating history adjustment graphs of the injectivity index (pL=8.0) with an increase in the area open to the flow for a well in a carbonate reservoir, being (a) for an area of 50 m²; and (b) for an area of 200 m².

FIG. 7 illustrating a graph of history adjustment of the injectivity index (pL=7.0) with an increase in the area open to the flow to another well in a carbonate reservoir, being (a) for an area of 34 m²; and (b) for an area of 350 m².

### DETAILED DESCRIPTION OF THE INVENTION

The combined experimental and simulation method for estimating the loss of injectivity in carbonate reservoirs, considering reactive effects and presence of suspended solids according to the present invention, comprises the following steps:

### a) Experimental Method

a.1) Saturate the rock samples (plugs) with a non-reactive water—NRW;
a.2) Confine at a pressure equal to the confinement pressure to which they were subjected in routine basic petrophysics tests, in this case at 1000 psi (6.895 MPa). The confining pressure value must not be greater than the stress values to which the samples were originally subjected. The objective is to prevent the sample from being damaged during this step. Thus, the maximum limit will depend on each sample and its origin. Confinement should be performed with a gradual increase in pressure (steps of 500 psi (3.447 MPa));
a.3) After the system is pressurized, carry out a flow with the non-reactive water to check the initial water permeability and pH, under conditions of room temperature, system pressure equal to 1000 psi (6.895 MPa) and injection flow rate of 0.8 ml/min The pressure will always be equal to the confining pressure to which they were submitted in the routine tests of basic petrophysics and the flow rate can be up to 2 ml/min;
a.4) Next, inject about 900 mL of desulfated seawater (DSW)+acid+solids, where the organic acid added to the water is at a concentration of 0.06 mol/L of formic acid and 0.34 mol/L of sodium formiate to reach a solution at pH=4.2; or nitric acid is added to the water in the amount necessary to reach pH=3.0 (concentration of nitric acid in water equal to 0.001 mol/L). The fluid addition flow rate is 0.8 ml/min; the solids added to the water are at a concentration of 20 mg/L of solids, and this value may vary depending on the concentration of solids to be simulated in the injected fluid, remembering that the effective value was 10% of this value. The solid used is a pulverized sandstone with a size between 45 and 75 μm. The greater the volume injected, the greater the effects of chemical reactions on the samples;
a.5) Carry out a filtration in a 0.45 μm mixed cellulose ester membrane at the point where the plug is located to determine the effective value of the suspended solids content;
a.6) Collect samples at the system outlet, after the fluid has come into contact with the rock, to monitor changes in chemical composition;
a.7) Determine the concentration of sodium, calcium, magnesium and potassium by the atomic emission spectrometry technique (ICP-OES), and of chloride, analyzed by titration; and also measure the pH of the solution at the test outlet under ambient conditions.

### b) Simulation Method

b.1) Determine the parameter pL of the Perkins and Gonzalez modeling with the data from the experimental test (during the test the flow and pressure values are measured and, from these, the resistance to flow is calculated. The parameter pL is the angular coefficient cologarithm obtained by plotting this flow resistance as a function of the accumulated injected volume—measured in the test—divided by the area open to the flow—determined from the sample diameter) or with history adjustment of the injectivity index of the well with injection of desulphated seawater (for this adjustment, the pressure and flow rate data measured in the field are considered). From these data, it is possible to determine the variation of the injectivity index over time and, with the Perkins & Gonzalez modeling, to determine the value of the parameter pL);
b.2) Carry out the adjustment of the open area to the flow of the Perkins and Gonzalez modeling according to the pL determined in item b.1 (comparison with the reactive and non-reactive test in the presence of suspended solids) or with the history adjustment of the injectivity index of item b.1. The value of the parameter pL, which is a function of the water and rock quality, must be the same in the reactive and non-reactive cases. What changes in the reactive scenario is the area open to the flow, which is increased by dissolution reactions. Knowing the value pL in a non-reactive scenario, it is possible to obtain the real area in the reactive case using the Perkins & Gonzalez model, the value pL is fixed and the area is modified to guarantee the adjustment of the obtained experimental data;
b.3) Carry out the Perkins and Gonzalez simulation to predict the injectivity index over time, also using well parameters and operating data, in addition to the area value adjusted in item b.2. The great relevance of the methodology is the determination of the future behavior of the wells, being possible to determine the need for interventions. By ignoring the reactivity, the appropriate adjustments are not made in the area and the simulations become more pessimistic. With the data obtained in the laboratory, it is possible to obtain both the pL, which will be mainly related to water quality, and the area adjustment necessary to represent the chemical reactivity. Thus, better predictions are obtained.

### EXAMPLES

The following examples are presented in order to more fully illustrate the nature of the present invention and the manner of practicing the same, without, however, being considered as limiting its content.

From an experimental test, the determination of the parameter pL of the Perkins and Gonzalez modeling is made. This parameter is related to water and rock quality. The test is done from the flow of a reactive fluid with the presence of solids in rock samples. With the parameter determined, the open area to the flow is adjusted and the injectivity index is predicted over time, also using well parameters and operation data. As the Perkins and Gonzalez model does not predict the reactivity of carbonate systems, it was necessary to develop an experimental methodology and a simulation method to alter the area open to the flow. This method also includes evaluation of the injectivity history of analogous fields.

The method of the present invention was applied to predict the loss of injectivity in the scenario of reinjection of water produced in a carbonate reservoir. This prediction considered the reactivity of the carbonate reservoir and the presence of suspended solids and oil in the injected water and was implemented in the production curves of the oil field.

### Example 1: Experimental Method

A reactive flow was used through the injection of acid, since a reactive fluid with a mixture of water+CO₂ causes infeasibility of adding solids in pressurized bottles. Thus, the test conditions were optimized to achieve a dissolution similar to that found in the tests using CO₂, that is, dissolution with formation of channels in the plug and not a homogeneous dissolution. In addition to the concentration and type of acid, the injection rate greatly influences this dissolution.

Two types of acid were tested: HNO₃ (strong acid) and formic acid (weak acid). The initial acid concentration and pH were estimated from the CurTiPo™ spreadsheet (available at: http://www.iq.usp.br/gutz/Curtipot.html) considering a dissolution similar to that performed in the reactive flow tests with CO₂.

For the formation of the channel in the samples, a low injection flow rate was used. Another aspect that impacted the choice of this flow rate was the permanence of suspended solids in solution during the test period. A flow rate equal to 0.8 ml/min was chosen to meet the criteria described above.

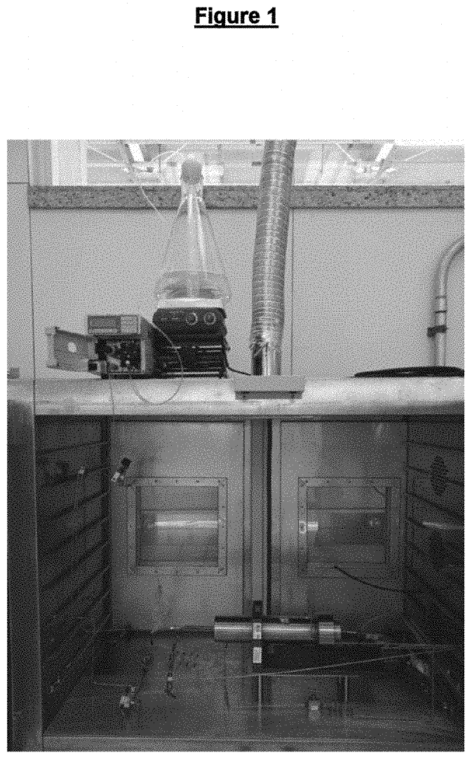

FIG. 1 shows the flow system used in the tests. Initially, the plugs were saturated with a non-reactive water—NRW (Table 1). These plugs were then confined to a pressure equal to the confining pressure to which they were subjected in routine basic petrophysics tests. While the entire system was pressurized with non-reactive water to the test pressure, the confining pressure was increased in steps of 500 psi (3.447 MPa). A back pressure valve was used to maintain the system pressure.

With the system ready and pressurized, a flow was carried out with the non-reactive water in order to verify the permeability to water and the initial pH. The experimental conditions were: ambient temperature, system pressure equal to 1000 psi (6.895 MPa) and injection flow rate of 0.8 ml/min.

TABLE 1
Composition of non-reactive water (NRW) used in the tests.

| Component | Concentration (mg/L) |
|---|---|
| Na⁺ | 32311 |
| Ca⁺² | 213 |
| Mg²⁺ | 74 |
| Cl⁻ | 50386 |
| HCO₃⁻ | 57 |

Next, about 900 mL of water+acid+solids were injected. The composition of the water is shown in Table 2 and has been synthesized to represent the injection of desulfated seawater (DSW).

For the tests carried out with organic acid, 0.06 mol/L of formic acid and 0.34 mol/L of sodium formiate were added to the water, resulting in a solution at pH=4.2. For the tests carried out with nitric acid, this was added to the water in the amount necessary to reach pH=3.0.

The solid added to the water was a pulverized sandstone with a size between 45 and 75 μm. Initially, a concentration of 20 mg/L of solids was added to the water. However, due to the decantation of these particles in the system, there is a reduction in the concentration that actually reaches the plug. To confirm the concentration of solids effectively used in the test, a membrane filtration of 0.45 μm cellulose mixed ester was performed at the point where the plug would be. On average, the effective concentration of suspended solids was 2 mg/L in the tests.

TABLE 2
Composition of injected water (DSW) used in the tests.

| Component | Concentration (mg/L) |
|---|---|
| Na⁺ | 11400 |
| Ca⁺² | 10 |
| Mg²⁺ | 72 |
| K⁺ | 442 |
| Cl⁻ | 18250 |
| HCO₃⁻ | 66 |
| SO₄⁻ | 4 |

Throughout the test, samples were collected at the outlet of the system, after the fluid had come into contact with the rock, to monitor possible changes in composition. The concentrations of sodium, calcium, magnesium and potassium were determined, analyzed by the atomic emission spectrometry technique (ICP-OES), and of chloride, analyzed by titration. In some samples, the pH of the solution at the test outlet was also measured under ambient conditions. In addition, the pressure difference at the inlet and outlet of the plug was recorded, making it possible to calculate the permeability throughout the test.

Five experiments were performed. The objective of these tests was to evaluate the effects of carbonate dissolution and loss of injectivity concomitantly. Tests TF01-2019 and TF03-2019 were performed with formic acid at pH=4.2. In the second test, there was further the addition of solids. The tests TF04-2019 and TF05-2019 were performed with nitric acid at pH=3.0, and the addition of solids took place in the second test (TF05-2019). The TF02-2019 was performed only with solids for comparison with the other tests performed with acid. Table 3 presents a summary of this experimental design.

TABLE 3
Experimental design.

| | Carbonate | | Presence |
|---|---|---|---|
| Test | Sample Code | Injection Fluid | of solids |
| TF01-2019 | I05-2018 | DSW + Formic Acid + Sodium | No |
| | | Formiate at pH = 4.2 | |
| TF02-2019 | I01-2018 | NRW | Yes |
| TF03-2019 | I02-2018 | DSW + Formic Acid + Sodium | Yes |
| | | Formiate at pH = 4.2 | |
| TF04-2019 | I04-2018 | DSW + Nitric Acid at pH = 3.0 | No |
| TF05-2019 | I03-2018 | DSW + Nitric Acid at pH = 3.0 | Yes |

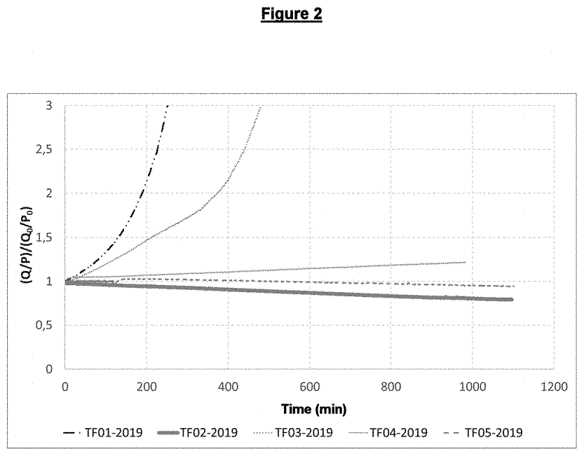

FIG. 2 presents the (Q/P)/(Q₀/P₀) data over time for all the tests described above. This graph gives us an idea of the normalized injectivity index for each test.

For the tests performed with the formic buffer (TF01-2019 and TF03-2019), the values of (Q/P)/(Q₀/P₀) increased. However, it can be seen that, in the presence of solids, this increase was smaller, since in addition to the positive effect of the dissolution, there was the effect of the damage caused by the solids. In both tests, however, the effect of dissolution was predominant, mainly due to channel formation in the sample, leading to an increase in permeability and, consequently, a gain in injectivity.

For the tests performed with nitric acid (TF04-2019 and TF05-2019), the effect of damage caused by the presence of solids was preponderant, since the reactivity, in this case, was lower. In the test TF04-2019, there was a gain in injectivity, but much smaller when compared to the TF01-2019 (with the addition of the formic buffer).

The test TF02-2019 was performed taking into account only the damage effect caused by solids. In this case, a loss of injectivity was observed over time, as expected. Comparing with the test TF05-2019, it can be seen that the loss of injectivity caused by solids can be mitigated in scenarios of reactivity between the reservoir rock and the injected fluid.

In this way, it is important to note that the preponderant effect (gain or loss of injectivity) depends on the intensity of the carbonate dissolution reaction and the quality of the water in terms of suspended solids content. However, not considering the rock-fluid interaction effect for carbonate reservoir scenarios will always lead to very pessimistic loss of injectivity predictions.

### Example 2: Simulation Method

The model used for the predictions of loss of injectivity is the one proposed by PERKINS, T. K. & GONZALEZ, J. A. (1985) “The effect of thermoelastic stresses on injection well fracturing”, Society of Petroleum Engineers Journal, USA, SPE11332. This model considers that the pressure increase due to damage formation is given by equation 03.

$$Δ {P}_{s} = \frac{{i}_{w} {μ}_{w} {R}_{s}}{{A}_{f}} \qquad ( 03 )$$

### Where:

Pₛ: pressure differential due to damage formation;
i_{w}: water injection flow rate;
μ_{w}: water viscosity;
Rₛ: flow resistance caused by damage;
A_{f}: area in which the solids present in the injection water will be deposited, forming the damage.

The value of Rₛ is calculated by equation 04.

$${R}_{s} = \frac{λ {W}_{i}}{A} \qquad ( 04 )$$

### Where:

λ: water and reservoir quality factor;
Wᵢ: accumulated volume of injected water;
A: area open to the flow.

The factor pL is defined as the cologarithm of λ (equation 05) from injection water displacement tests in rock plugs that represent the reservoir or history adjustment of the injectivity index, with its value related with the quality of water and rock. Low values (around 2) mean high injectivity losses (greater damage) while higher values (around 8) indicate more adequate injection conditions.

pL=−log(λ) (05)

The described modeling considers only injectivity loss due to the quality characteristics of the injected water and the reservoir. Therefore, it does not consider the effect of injectivity gains due to reactivity. In this way, the Perkins and Gonzalez modeling does not allow adjusting the data history of carbonate reservoir wells. Even considering the best water quality (pL=8), the model sees in the maximum the maintenance of the injectivity index. In some cases, to keep the injectivity index constant, and considering pL=8, it is necessary to increase the area open to the flow, in order to adjust the observed data.

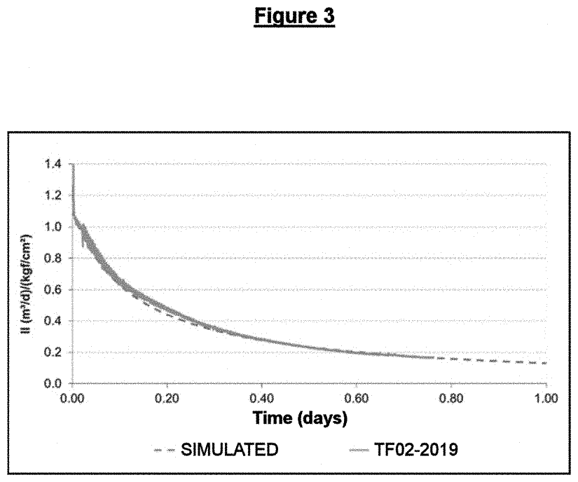

This Perkins and Gonzalez modeling was used to compare tests TF02-2019 (NRW+solids) and TF05-2019 (DSW+nitric acid+solids). FIG. 3 shows the adjustment performed with the data from the test TF02-2019. The value for the parameter pL was equal to 4.25.

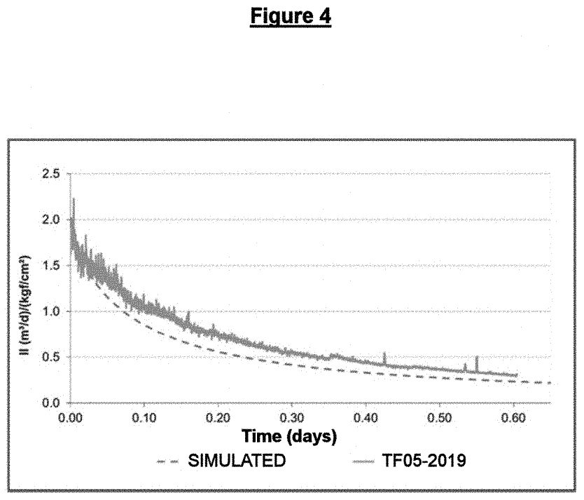

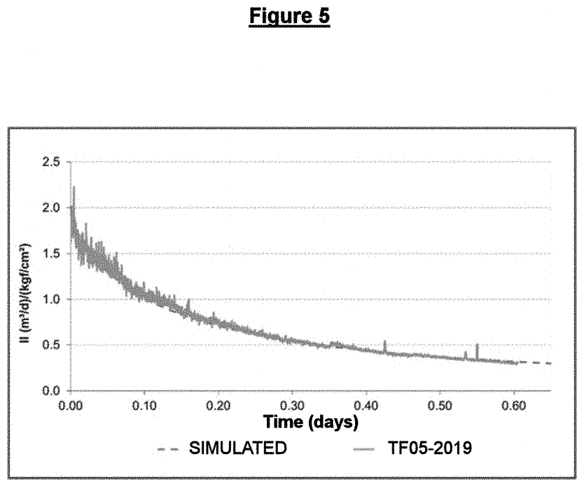

As the TF05-2019 was carried out with the same concentration of solids in the water and with a plug from the same outcrop, the same value pL adjusted above can be used. FIG. 4 shows the result of this simulation. It is observed that the experimental result was better than the simulated one. In fact, as there was an addition of acid and, consequently, a dissolution reaction of calcite, it can be said that the area open to the flow, which is a parameter informed for the simulation, was changed. Returning to the simulation and increasing the area, it was possible to perform the curve fitting to the experimental data. The area has increased by 20% of the original value. It should be noted that the test took place in a short period of time. Thus, the loss of injectivity in the presence of solids ends up being attenuated. For longer periods, the area increase should be greater. FIG. 5 presents this adjustment for the test TF05-2019.

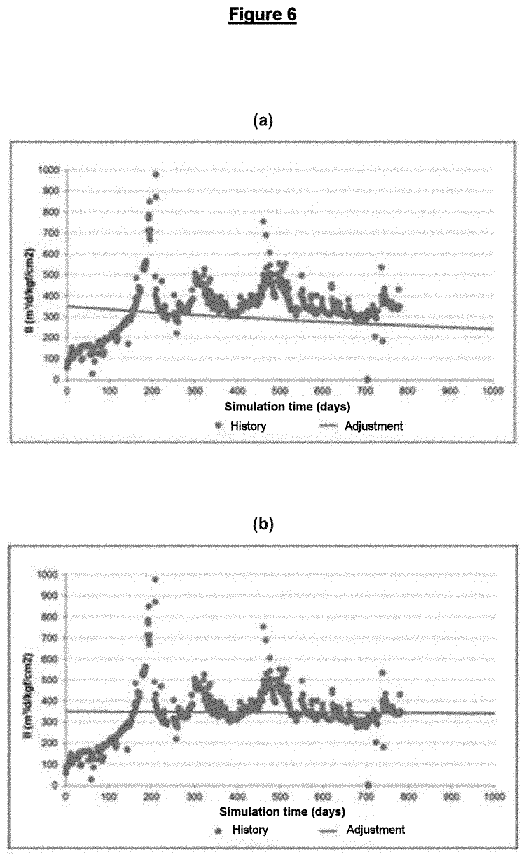

This method is also composed by the analysis of the behavior of the injectivity index in analogous fields. FIG. 6 presents a history adjustment for a carbonate reservoir well. The prediction of the Perkins and Gonzalez model is the straight line in both cases. The curve represents history data. For the 50 m² area, the expected behavior is much worse than that observed. Increasing the area by 4 times, the model limit is reached: constant injectivity.

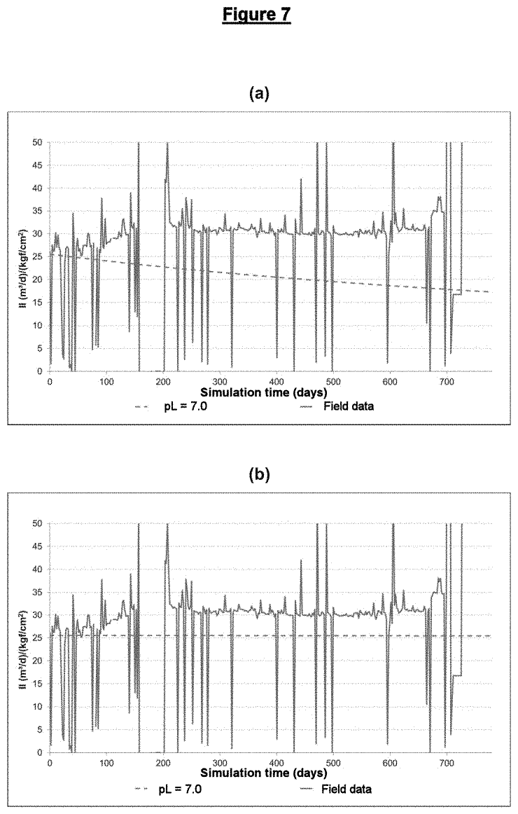

FIG. 7 presents another history adjustment for a carbonate reservoir well. The model prediction is the straight line in both cases. The curve represents history data. For the 34 m² area, the expected behavior is much worse than that observed. Increasing the area by 10 times, we arrive at the model's limit: constant injectivity.

It should be noted that, although the present invention has been described in relation to the attached drawings, it may undergo modifications and adaptations by technicians skilled on the subject, depending on the specific situation, but provided that it is within the inventive scope defined herein.

1- A COMBINED METHOD FOR ESTIMATING THE LOSS OF INJECTIVITY IN CARBONATE RESERVOIRS, considering reactive effects and the presence of suspended solids, characterized in that it comprises the following steps:
a) Experimental method:
a.1) Saturate the rock samples (plugs) with a non-reactive water—NRW;
a.2) Gradually increase the pressure and confine the system to a pressure equal to the confinement pressure to which they were submitted in the routine tests of basic petrophysics;
a.3) After the system is pressurized, carry out a flow with the non-reactive water to check the initial water permeability and pH, under conditions of room temperature and injection flow rate of 0.8 to 2.0 ml/min;
a.4) Next, inject about 900 mL of desulfated seawater (DSW)+acid+solids, where the fluid addition flow rate is 0.8 ml/min;
a.5) Carry out a filtration in a 0.45 μm mixed cellulose ester membrane at the point where the plug is located to determine the effective value of the suspended solids content;
a.6) Collect samples at the system outlet, after the fluid has come into contact with the rock, to monitor changes in chemical composition;
a.7) Determine the concentration of sodium, calcium, magnesium and potassium by the atomic emission spectrometry technique (ICP-OES), and of chloride, analyzed by titration; and also measure the pH of the solution at the test outlet under ambient conditions;
b) Simulation method:
b.1) Determine the parameter pL of the Perkins and Gonzalez modeling with data from the experimental test;
b.2) Carry out the adjustment of the open area to the flow of the Perkins and Gonzalez modeling according to the pL determined in item b.1 (comparison with the reactive and non-reactive test in the presence of suspended solids) or with the history adjustment the injectivity index of item b.1;
b.3) Carry out the Perkins and Gonzalez simulation to predict the injectivity index over time, also using well parameters and operating data, in addition to the area value adjusted in item b.2.

2- THE METHOD according to claim 1, characterized in that, in step a.2, the pressure is raised to 1000 psi (6.895 MPa), in steps of 500 psi (3.447 MPa).

3- THE METHOD according to claim 1, characterized in that the organic acid of step a.4 is formic acid with sodium formiate, with a concentration of 0.06 mol/L and 0.34 mol/L, respectively, to achieve a pH of 4.2.

4- THE METHOD according to claim 1, characterized in that the strong acid of step a.4 is nitric acid in the amount necessary to reach a pH of 3.0 (concentration of nitric acid in water equal to 0.001 mol/L).

5- THE METHOD according to claim 1, characterized in that the concentration of solids added to desulfated seawater (DSW) in step a.4 is from 2 to 20 mg/L.

6- THE METHOD according to claim 5, characterized in that the solids are a pulverized sandstone with a size between 45 and 75 μm.
